(19) United States
(12) Patent Application Publication
(10) Pub. No.: US 2026/0083390 A1
(43) Pub. Date: Mar. 26, 2026

(54) WEARABLE DIAPHRAGMATIC EFFICIENCY MONITORING DEVICES

(71) Applicant: SAN DIEGO STATE UNIVERSITY (SDSU) FOUNDATION, DBA SAN DIEGO STATE UNIVERSITY RESEARCH FOUNDATION, San Diego, CA (US)

(72) Inventor: Hakan Toreyin, San Diego, CA (US)

(21) Appl. No.: 19/338,719

(22) Filed: Sep. 24, 2025

Related U.S. Application Data
(60) Provisional application No. 63/698,357, filed on Sep. 24, 2024.

(51) Int. Cl. A61B 5/389; A61B 5/00; A61B 5/256; A61B 5/257

(52) U.S. Cl. CPC A61B 5/389; A61B 5/0022; A61B 5/256; A61B 5/257

(57) ABSTRACT

In alternative embodiments, provided are wearable diaphragmatic efficiency monitoring methods, systems, kits, and devices for measuring and monitoring diaphragmatic efficiency, and in alternative embodiments, allowing local respiratory monitoring of people with respiratory diseases or conditions, including for example cervical spinal cord injury (CSCI), asthma, sleep apnea, chronic obstructive pulmonary disease (COPD), patients undergoing positive airway pressure (PAP) testing, or PAP-NAP studies.

### PRIORITY CLAIM

This application claims the benefit of U.S. Provisional Application Ser. No. 63/698,357, filed Sep. 24, 2024, under 35 U.S.C. 119, which application is incorporated in its entirety by reference herein.

### STATEMENT AS TO FEDERALLY SPONSORED RESEARCH

This invention was made with government support under 5P2CHD101899-04 awarded by the National Institutes of Health. The government has certain rights in the invention.

### TECHNICAL FIELD

This disclosure generally relates to respiratory technology. In alternative embodiments, provided are wearable diaphragmatic efficiency monitoring methods, systems, and devices that may be used to measure and monitor diaphragmatic efficiency, and in alternative embodiments, may allow mobile or in-home respiratory monitoring of people with respiratory diseases or conditions, including, for example, cervical spinal cord injury (CSCI), asthma, sleep apnea, patients undergoing positive airway pressure (PAP) testing, chronic obstructive pulmonary disease (COPD), or PAP-NAP studies. In alternative embodiments, provided are products of manufacture and kits, comprising portable, wearable diaphragmatic efficiency monitoring devices, and methods for using them.

### BACKGROUND

Respiratory dysfunction is a major contributor to morbidity and mortality across many high-risk clinical populations. For example, currently there are ˜18,000 new traumatic spinal cord injury (tSCI) cases yearly and ˜308,000 people living with tSCI in the United States [1]. Additionally, there is an even greater incidence and prevalence of nontraumatic SCI (ntSCI) [2]. Since 2015, tetraplegia from cervical spinal cord injury (CSCI) has constituted 59.6% of all tSCI, with a similar majority seen in ntSCI [1]. Chronic obstructive pulmonary disease (COPD), particularly in its severe stages (GOLD III and IV), is one of the most common causes of disability and death worldwide, affecting ˜6% of adults in the United States [3].

Despite their etiological differences, these conditions share a common pathophysiological burden: impaired diaphragmatic function [4], [5], [6] and reduced neuromechanical ventilatory capacity [7], [8], [9]. This results in compromised respiratory reserve and elevated risk for complications such as pneumonia, atelectasis, nocturnal hypoventilation, and respiratory failure, often progressing silently and remaining undetected until advanced stages.

The phrenic nerve originates from the third to fifth cervical roots and innervates the diaphragm. Diaphragm dysfunction occurs in individuals with CSCI due to disruption at these levels [14], and places them at elevated risk for serious respiratory complications, including pneumonia, atelectasis, and respiratory failure, due to reduced physiological reserve and diminished ability to compensate for respiratory stressors [17], [18], [19], [20]. Respiratory complications from SCI are most prevalent within the first year of injury but continue to occur throughout life [17].

In COPD, diaphragmatic inefficiency is primarily driven by lung hyperinflation, which flattens the diaphragm and reduces its mechanical advantage, compounded by increased work of breathing and air trapping [10], [11], [12]. This dysfunction is most pronounced in GOLD stage III/IV disease and is often accompanied by dynamic hyperinflation [13], [14], muscle fatigue [15], [16], and reduced ventilatory responsiveness during sleep and exertion [17].

Across all of these populations, respiratory decline is often silent, progressive, and episodic. Complications tend to worsen during high-risk periods [18], [19], [20], [21], including the onset of acute illness (e.g., viral infections, bronchitis, pneumonia) and the post-hospital recovery phase (e.g., following surgery or exacerbation), when individuals are especially vulnerable to secondary complications and re-hospitalization.

Despite their high risk, individuals affected by these conditions rarely receive continuous monitoring of respiratory effort, diaphragmatic performance, or ventilatory reserve outside of clinical environments. Accordingly, there is an urgent need for an unobtrusive, reliable, and physiologically meaningful wearable system to support early detection and intervention.

A reliable home-based monitoring system or otherwise local to the individual in need could add critical value across all phases of care, particularly during acute illness and post-hospital recovery, when individuals with CSCI or severe COPD are most at risk for respiratory decompensation. Early in a respiratory infection, continuous monitoring could detect signs of compensation or decline, enabling timely intervention. During recovery from pneumonia, surgery, or exacerbations, such monitoring could track respiratory stability, support reconditioning, and/or provide clinicians with objective insight into a patient's progress outside clinical settings. Beyond these critical episodes, a wearable system could also support therapeutic engagement. During underused interventions like inspiratory muscle training and pulmonary rehabilitation, despite strong evidence of benefit [22], [23], [24], [25], real-time feedback on respiratory effort and trends could improve motivation and adherence. The same platform could also serve as a general-purpose respiratory biomarker tool, enabling objective, longitudinal monitoring to support clinical trials of emerging therapies.

Despite such broad utility, there are currently no effective systems for comprehensive, passive respiratory monitoring that capture meaningful neuromuscular activity outside of clinical settings. Traditional tools such as handheld spirometers and peak flow meters can measure basic pulmonary output like airflow and volume [26], but they require deliberate effort and technique, limiting their use in individuals with impaired mobility, respiratory fatigue, or neuromuscular degeneration [27], [28], [29]. Commercial wearables (e.g., smartwatches, rings, pulse oximeters) provide partial proxies for respiratory status, typically limited to SpO₂ and respiratory rate [30], but their accuracy declines during motion [31], and they offer no insight into respiratory effort, neural drive, or muscle activation, which are crucial in understanding disease progression and compensatory breathing.

This technological gap leaves individuals with CSCI and severe COPD unmonitored during periods of elevated risk, with no alternative to burdensome in-person assessments such as spirometry, polysomnography (PSG), or diaphragm ultrasound. These visits are often logistically challenging, physically demanding, and episodic, and thus failing to capture day-to-day fluctuations or early warning signs.

Thus, there is a pressing need for an accurate, unobtrusive, and easy-to-use home monitoring system that can provide one or more of the following benefits: continuously capturing both respiratory mechanics and neuromuscular effort; functioning reliably across diverse real-world conditions (such as sleep, movement, and varying postures); and/or providing clinicians with actionable, high-resolution physiological data to support timely and informed care decisions.

### REFERENCES

The following references are provided herein for further understanding by those in the art. Citation of these references is not an admission that any of these references is prior art.
[1] National Spinal Cord Injury Statistical Center, “Traumatic Spinal Cord Injury Facts and Figures at a Glance.” Birmingham, AL: University of Alabama at Birmingham, 2023.
[2] S. W. Lee, et al., “Clinical characteristics, hospital course, and disposition of patients with nontraumatic spinal cord injury in a large private health care system in the United States,” The Journal of Spinal Cord Medicine, vol. 0, no. 0, pp. 1-10, May 2022, doi: 10.1080/10790268.2022.2069533.
[3] CDC, “National Trends in COPD,” Chronic Obstructive Pulmonary Disease (COPD). Accessed: Jul. 5, 2025. [Online]. Available: https://www.cdc.gov/copd/php/case-reporting/national-trends-in-copd.html
[4] M.-H. Wen and K.-Z. Lee, “Diaphragm and Intercostal Muscle Activity after Mid-Cervical Spinal Cord Contusion in the Rat,” J. Neurotrauma, vol. 35, no. 3, pp. 533-547, February 2018, doi: 10.1089/neu.2017.5128.
[5] L. M. Mercier et al., “Intraspinal microstimulation and diaphragm activation after cervical spinal cord injury,” J. Neurophysiol., vol. 117, no. 2, pp. 767-776, February 2017, doi: 10.1152/jn.00721.2016.
[6] D. K. Mckenzie, J. E. Butler, and S. C. Gandevia, “Respiratory muscle function and activation in chronic obstructive pulmonary disease,” J. Appl. Physiol., vol. 107, no. 2, pp. 621-629, August 2009, doi: 10.1152/japplphysiol.00163.2009.
[7] N. J. Tester, D. D. Fuller, J. S. Fromm, M. R. Spiess, A. L. Behrman, and J. H. Mateika, “Long-Term Facilitation of Ventilation in Humans with Chronic Spinal Cord Injury,” Am. J. Respir. Crit. Care Med., vol. 189, no. 1, pp. 57-65, January 2014, doi: 10.1164/rccm.201305-0848OC.
[8] D. E. O'Donnell, “Ventilatory limitations in chronic obstructive pulmonary disease,” Med. Sci. Sports Exerc., vol. 33, no. 7 Suppl, pp. S647-655, July 2001, doi: 10.1097/00005768-200107001-00002.
[9] D. E. O'Donnell, J. A. Guenette, F. Maltais, and K. A. Webb, “Decline of Resting Inspiratory Capacity in COPD: The Impact on Breathing Pattern, Dyspnea, and Ventilatory Capacity During Exercise,” Chest, vol. 141, no. 3, pp. 753-762, March 2012, doi: 10.1378/chest.11-0787.
[10] N. Scheibe, N. Sosnowski, A. Pinkhasik, S. Vonderbank, and A. Bastian, “Sonographic evaluation of diaphragmatic dysfunction in COPD patients,” Int. J. Chron. Obstruct. Pulmon. Dis., vol. 10, pp. 1925-1930 September 2015, doi: 10.2147/COPD.S85659.
[11] D. E. O'Donnell and P. Laveneziana, “Dyspnea and Activity Limitation in COPD: Mechanical Factors,” COPD J. Chronic Obstr. Pulm. Dis., vol. 4, no. 3, pp. 225-236, January 2007, doi: 10.1080/15412550701480455.
[12] F. Laghi and M. J. Tobin, “Disorders of the Respiratory Muscles,” Am. J. Respir. Crit. Care Med., vol. 168, no. 1, pp. 10-48, July 2003, doi: 10.1164/rccm.2206020.
[13] D. E. O'Donnell, J. A. Neder, and A. F. Elbehairy, “Physiological impairment in mild COPD,” Respirology, vol. 21, no. 2, pp. 211-223, 2016, doi: 10.1111/resp.12619.
[14] D. E. O'Donnell and K. B. Gebke, “Activity restriction in mild COPD: a challenging clinical problem,” Int. J. Chron. Obstruct. Pulmon. Dis., vol. 9, pp. 577-588, June 2014, doi: 10.2147/COPD.S62766.
[15] M. Jeffery Mador, T. J. Kufel, L. A. Pineda, and G. K. Sharma, “Diaphragmatic Fatigue and High-intensity Exercise in Patients with Chronic Obstructive Pulmonary Disease,” Am. J. Respir. Crit. Care Med., vol. 161, no. 1, pp. 118-123, January 2000, doi: 10.1164/ajrccm.161.1.9903010.
[16] M. I. Polkey and J. Moxham, “Attacking the disease spiral in chronic obstructive pulmonary disease,” Clin. Med., vol. 6, no. 2, pp. 190-196, March 2006, doi: 10.7861/clinmedicine.6-2-190.
[17] F. Maltais, A. A. Simard, C. Simard, J. Jobin, P. Desgagnés, and P. LeBlanc, “Oxidative capacity of the skeletal muscle and lactic acid kinetics during exercise in normal subjects and in patients with COPD.,” Am. J. Respir. Crit. Care Med., vol. 153, no. 1, pp. 288-293, January 1996, doi: 10.1164/ajrccm.153.1.8542131.
[18] K. Luo et al., “Risk Factors and Nomogram for Postoperative Pulmonary Infection in Patients with Cervical Spinal Cord Injury,” World Neurosurg., vol. 177, pp. e317-e324, September 2023, doi: 10.1016/j.wneu.2023.06.040.
[19] K. S. Held, O. Steward, C. Blanc, and T. E. Lane, “Impaired immune responses following spinal cord injury lead to reduced ability to control viral infection,” Exp. Neurol., vol. 226, no. 1, pp. 242-253, November 2010, doi: 10.1016/j.expneurol.2010.08.036.
[20] S. Suissa, S. Dell′Aniello, and P. Ernst, “Long-term natural history of chronic obstructive pulmonary disease: severe exacerbations and mortality,” Thorax, vol. 67, no. 11, pp. 957-963, November 2012, doi: 10.1136/thoraxjnl-2011-201518.
[21] T. M. A. Wilkinson, G. C. Donaldson, J. R. Hurst, T. A. R. Seemungal, and J. A. Wedzicha, “Early Therapy Improves Outcomes of Exacerbations of Chronic Obstructive Pulmonary Disease,” Am. J. Respir. Crit. Care Med., vol. 169, no. 12, pp. 1298-133 June 2004, doi: 10.1164/rccm.200310-1443OC.
[22] C. R. West, B. J. Taylor, I. G. Campbell, and L. M. Romer, “Effects of inspiratory muscle training on exercise responses in Paralympic athletes with cervical spinal cord injury,” Scand. J. Med. Sci. Sports, vol. 24, no. 5, pp. 764-772, 2014, doi: 10.1111/sms.12070.
[23] T. McDonald and K. and Stiller, “Inspiratory muscle training is feasible and safe for patients with acute spinal cord injury,” J. Spinal Cord Med., vol. 42, no. 2, pp. 220-227, March 2019, doi: 10.1080/10790268.2018.1432307.
[24] A. Woods, Gustafson,Owen, Williams,Mark, and R. and Stiger, “The effects of inspiratory muscle training on inspiratory muscle strength, lung function and quality of life in adults with spinal cord injuries: a systematic review and Meta-analysis,” Disabil. Rehabil., vol. 45, no. 17, pp. 2703-2714 August 2023, doi: 10.1080/09638288.2022.2107085.
[25] D. J. Berlowitz and J. Tamplin, “Respiratory muscle training for cervical spinal cord injury-Berlowitz, DJ-2013|Cochrane Library”, Accessed: Jul. 16, 2025. [Online]. Available: https://www.cochranelibrary.com/cdsr/doi/10.1002/14651858.CD008507.pub2/full.
[26] R. G. Barr et al., “Reproducibility and Validity of a Handheld Spirometer,” Respir. Care, vol. 53, no. 4, pp. 433-441, April 2008, doi: 10.4187/respcare.08530433.
[27] T. M. Maher et al., “A review of the challenges, learnings and future directions of home handheld spirometry in interstitial lung disease,” Respir. Res., vol. 23, no. 1, p. 307, November 2022, doi: 10.1186/s12931-022-02221-4.
[28] S. Sarfaraz, Z. Sund, and N. Jarad, “Real-time, once-daily monitoring of symptoms and FEV1 in cystic fibrosis patients-A feasibility study using a novel device,” Clin. Respir. J., vol. 4, no. 2, pp. 74-82, 2010, doi: 10.1111/j.1752-699X.2009.00147.x.
[29] S. Komal et al., “Utility of FEV1/FEV6 index in patients with multimorbidity hospitalized for decompensation of chronic diseases,” PLOS ONE, vol. 14, no. 8, p. e0220491, August 2019, doi: 10.1371/journal.pone.0220491.
[30] S. D. Bergese et al., “Multicenter Study Validating Accuracy of a Continuous Respiratory Rate Measurement Derived From Pulse Oximetry: A Comparison With Capnography,” Anesth. Analg., vol. 124, no. 4, pp. 1153-1159 April 2017, doi: 10.1213/ANE.0000000000001852.
[31] E. A. Jafleh, F. A. Alnaqbi, H. A. Almaeeni, S. Faqeeh, M. A. Alzaabi, and K. Al Zaman, “The Role of Wearable Devices in Chronic Disease Monitoring and Patient Care: A Comprehensive Review,” Cureus, vol. 16, no. 9, p. e68921, September 2024, doi: 10.7759/cureus.68921.
[32] “React Native. Learn once, write anywhere.” Accessed: Jul. 5, 2025. [Online]. Available: https://reactnative.dev/
[33] S. Kano and H. Mekaru, “Preliminary comparison of respiratory signals using acceleration on neck and humidity in exhaled air,” Microsyst. Technol., vol. 27, no. 1, pp. 1-9, January 2021, doi: 10.1007/s00542-020-04889-4.
[34] M. Chu et al., “Respiration rate and volume measurements using wearable strain sensors,” Npj Digit. Med., vol. 2, no. 1, p. 8, February 2019, doi: 10.1038/s41746-019-0083-3.
[35] “AD5940 Datasheet and Product Info|Analog Devices.” https://www.analog.com/en/products/AD5940.html #product-overview (accessed Jun. 10, 2023).
[36] L. H. Hamilton, et al., “Impedance measurement of tidal volume and ventilation,” Journal of Applied Physiology, vol. 20, no. 3, pp. 565-568, May 1965, doi: 10.1152/jappl.1965.20.3.565.
[37] J. A. Berkebile, et al., “Towards Estimation of Tidal Volume and Respiratory Timings via Wearable-Patch-Based Impedance Pneumography in Ambulatory Settings,” IEEE Transactions on Biomedical Engineering, vol. 69, no. 6, pp. 1909-1919 June 2022, doi: 10.1109/TBME.2021.3130540.
[38] J. Rosell and J. G. Webster, “Signal-to-motion artifact ratio versus frequency for impedance pneumography,” IEEE Transactions on Biomedical Engineering, vol. 42, no. 3, pp. 321-323, March 1995, doi: 10.1109/10.364521.
[39] “ADS1198 data sheet, product information and support|TI.com.” https://www.ti.com/product/ADS1198 #features (accessed Jun. 10, 2023).
[40] S. Hersek, H. et al., “A Robust System for Longitudinal Knee Joint Edema and Blood Flow Assessment Based on Vector Bioimpedance Measurements,” IEEE Transactions on Biomedical Circuits and Systems, vol. 10, no. 3, pp. 545-555, June 2016, doi: 10.1109/TBCAS.2015.2487300.
[41] S. Shah, H. et al., “A proof-of-concept real-time processing to characterize vascular flow,” in 2022 44th Annual International Conference of the IEEE Engineering in Medicine & Biology Society (EMBC), July 2022, pp. 2912-2915. doi: 10.1109/EMBC48229.2022.9871424.
[42] P. Aqueveque et al., “Simple wireless impedance pneumography system for unobtrusive sensing of respiration,” Sensors, vol. 20, no. 18, p. 5228 September 2020, doi: 10.3390/s20185228.
[43] M. Klum, T. Schenck, A. Pielmus, T. Tigges, and R. Orglmeister, “Short distance impedance pneumography,” Current Directions Biomed. Eng., vol. 4, no. 1, pp. 109-113, September 2018, doi: 10.1515/cdbme-2018-0028.
[44] V. Jeyhani, T. Vuorinen, K. Noponen, M. Mäntysalo, and A. Vehkaoja, “Optimal short distance electrode locations for impedance pneumography measurement from the frontal thoracic area,” in Proc. 14th Medit. Conf. Med. Biol. Eng. Comput., E. Kyriacou, S. Christofides, and C. S. Pattichis, Eds., Cham, Switzerland: Springer, 2016, pp. 1144-1149, doi: 10.1007/978-3-319-32703-7_223.
[45] A. H. Jonkman et al., “Analysis and applications of respiratory surface EMG: Report of a round table meeting,” Crit. Care, vol. 28, no. 1, p. 2, January 2024, doi: 10.1186/s13054-023-04779-x.
[46] S. Kano and H. Mekaru, “Preliminary comparison of respiratory signals using acceleration on neck and humidity in exhaled air,” Microsyst. Technol., vol. 27, no. 1, pp. 1-9, January 2021, doi: 10.1007/s00542-020-04889-4.
[47] M. Chu et al., “Respiration rate and volume measurements using wearable strain sensors,” Npj Digit. Med., vol. 2, no. 1, p. 8, February 2019, doi: 10.1038/s41746-019-0083-3.

### SUMMARY

In alternative embodiments, provided are portable, e.g., wearable, diaphragmatic efficiency monitoring systems, methods, and devices that are used to measure and monitor diaphragmatic efficiency, and in alternative embodiments, allow local (e.g., mobile or in-home) respiratory monitoring of people with respiratory diseases or conditions, including for example cervical spinal cord injury (CSCI), chronic obstructive pulmonary disease (COPD), asthma, sleep apnea, patients undergoing positive airway pressure (PAP) testing, or PAP-NAP studies. In alternative embodiments, provided are products of manufacture and kits, comprising portable, wearable diaphragmatic efficiency monitoring devices, and methods for using them.

In alternative embodiments, provided are products of manufacture for measuring and monitoring diaphragmatic efficiency in an individual in need thereof, comprising a wearable hardware comprising noninvasive sensing devices, components, or modalities, the product of manufacture comprising:
(a) a hardware impedance pneumography (IP) device comprising one or a plurality of electrodes fabricated for attachment to the thorax (chest) of the individual in need thereof for producing IP signals,
wherein optionally the IP device further comprises a signal processor, e.g., a chip, for phase-sensitive detection for enhanced signal-to-noise-ratio (SNR) by amplifying a narrow bandwidth of the produced IP signals and suppressing out-of-band noise, and controlling injected current signal amplitude to meet one or more safety standards (e.g., IEC 60601-1) and optimize SNR within the dynamic range, wherein optionally a minimum detectable signal, e.g., of 55 μΩrms, can be detected;
(b) an inertial measurement unit (IMU) device comprising a miniaturized MEMS-based unit that integrates an accelerometer and a gyroscope, the accelerometer configured to serve as the primary sensor for capturing linear thoracic movement due to breathing, and the gyroscope configured to provide angular velocity data for distinguishing respiration-induced motion from postural shifts;
(c) a surface electromyography (sEMG) device comprising one or a plurality of electrodes fabricated for (operative) attachment to the individual in need thereof, optionally to a thorax (chest) and/or neck of the individual in need thereof for producing sEMG signals,
wherein optionally the sEMG device further comprises a signal processor such as a high common-mode rejection ratio instrumentation amplifier and gain controller to optimize sEMG signal amplification without saturation,
wherein the IP, IMU, and sEMG devices are operably connected to:
(i) a power source (optionally a battery) and power management electronics, and
(ii) a data transmittal device for transmitting IP, IMU, and sEMG device signals or diaphragmatic measurement data produced from such signals to a remote processor-based device, wherein optionally the remote processor-based device comprises a remote computer, cloud storage, tablet or cell phone.

In alternative embodiments of products of manufacture as provided herein:
the IMU device may be selected to achieve a resolution greater than 10 bits with a noise floor below 100 μg/√Hz and a full-scale range no greater than ±2 g, and, further, the IMU device may be ultra-low power while achieving the above specifications within a respiratory bandwidth of less than 3 Hz (e.g., ADXL326, BMI160) (Based on literature, e.g., [46], [47], respiratory-induced motion typically produces accelerations less than a few hundreds of milli-g range),
all or separately each of the product of manufacture components are part of, or adhered to (optionally glued to and/or physically attached to), a skin wearable patch; and/or
IP, IMU, and sEMG signals are linear filtered to suppress (or substantially suppress) noise sources, wherein optionally the noise sources comprise high-frequency noise, baseline wander and/or electrocardiogram interference.

In alternative embodiments, provided are methods for detecting and measuring and monitoring diaphragmatic efficiency in an individual in need thereof comprising:
(a) providing a product of manufacture as provided herein, and attaching electrodes to the individual in need thereof; and
(b) using the IP, IMU, and sEMG devices taking or reading diaphragmatic measurement data from the individual in need thereof, and transmitting the diaphragmatic measurement data to a remote processor-based device, wherein optionally the remote processor-based device comprises a remote computer, cloud storage, tablet, or cell phone.

In alternative embodiments of methods as provided herein:
the individual in need thereof has a respiratory disease or condition, or the individual in need thereof is a patient undergoing positive airway pressure (PAP) testing, or PAP-NAP studies;
the respiratory disease or condition comprises: a cervical spinal cord injury (CSCI), asthma, chronic obstructive pulmonary disease (COPD), and/or sleep apnea;
the method further comprises use of a Savitzky-Golay filter to clean the IP signal (and generate a cleaned IP signal) to mitigate anticipated cardiogenic oscillation interferences;
the method further comprises analyzing the cleaned IP signal to determine respiration phases, durations, respiration rates, and/or peak-to-peak amplitude changes; and/or,
the sEMG device further comprises a high common-mode rejection ratio instrumentation amplifier and gain controller to optimize sEMG signal amplification without saturation.

In alternative embodiments, provided are use of a product of manufacture of as provided herein for measuring and monitoring diaphragmatic efficiency in an individual in need thereof, wherein optionally the individual in need thereof has a respiratory disease or condition, or the individual in need thereof is a patient undergoing positive airway pressure (PAP) testing, or PAP-NAP studies, and optionally the respiratory disease or condition comprises: a cervical spinal cord injury (CSCI), asthma, chronic obstructive pulmonary disease (COPD), and/or sleep apnea.

In alternative embodiments, provided are products of manufacture of any of the above embodiments or combination for use in measuring and monitoring diaphragmatic efficiency in an individual in need thereof, wherein optionally the individual in need thereof has a respiratory disease or condition, or the individual in need thereof is a patient undergoing positive airway pressure (PAP) testing, or PAP-NAP studies, and optionally the respiratory disease or condition comprises: a cervical spinal cord injury (CSCI), asthma, chronic obstructive pulmonary disease (COPD), and/or sleep apnea.

In alternative embodiments, provided are kits comprising a product of manufacture as provided herein, and optionally further comprising leads for operatively connecting the product of manufacture to an individual in need thereof.

The details of one or more exemplary embodiments of the invention are set forth in the accompanying drawings and the description below. Other features, objects, and advantages of the invention will be apparent from the description and drawings, and from the claims.

All publications, patents, patent applications, American Type Culture Collection (ATCC) deposits and NCBI reference sequences cited herein are hereby expressly incorporated by reference in their entireties for all purposes.

### DESCRIPTION OF DRAWINGS

The drawings set forth herein are illustrative of exemplary embodiments provided herein and are not meant to limit the scope of the invention as encompassed by the claims.

FIG. 1 depicts a noninvasive wearable system according to an example embodiment.

FIG. 2 illustrates hardware modules for an example individual patch for the wearable system of FIG. 1.

FIG. 3 illustrates example positions of electrodes on the thorax of an individual for practicing or using a device as provided herein, where the electrodes are used in devices as provided herein.

FIG. 4 illustrates a sensor board for an example system for sEMG and IP sensing.

FIG. 5 illustrates top and side views of a hardware stack for an example system that integrates sEMG and IP sensing modalities, including 1) the sensor board of FIG. 4; 2) an IP microcontroller (IP-MCU) board; and 3) a data logger and power source.

FIG. 6 shows a circuit board illustrates an integration IP, sEMG, data acquisition, and power management electronics.

FIG. 7 shows example waveforms from an example signal processing chain, where IP waveforms (FIG. 7, left) were detrended using a second-order polynomial, and peak-to-peak amplitudes (IPₚₚ) were calculated. Instantaneous respiration rate (RRₘₑₐₛ) (FIG. 7, right) was determined from time differences between successive peaks. The IP panels show raw, detrended, and peak-detected signals. sEMG panels show raw, filtered, and SVD-cleaned signals with RMS (black) and peaks (red).

FIG. 8 shows scatter plots of TVₜᵣᵤₑ VS. IPₚₚ for subjects in experiments using example systems.

FIG. 9 shows differences between RRₘₑₐₛ and RRₜᵣᵤₑ across different positions in experiments, where black dashed line indicates mean difference, and red dashed lines indicate 95% confidence intervals.

FIG. 10 shows example simultaneous IP and raw sEMG data (uncorrected for ECG) from a subject using example hardware in experiments.

Like reference symbols in the various drawings indicate like elements.

### DETAILED DESCRIPTION

Individuals in need, e.g., patients, who fail to wean from mechanical ventilation exhibit increased activity in their diaphragm muscle electrical signals during spontaneous breathing; however, these patients are less effective at utilizing the increased diaphragm activity to generate sufficient tidal volume (TV), which represents the amount of air exchanged during each breath. Therefore, neuro-ventilatory efficiency (NVE) is a common measure of diaphragmatic efficiency that quantifies the conversion of electrical activity (EAdi) into inspired volume. As air enters the lungs, tissue conductivity decreases, leading to an increase in chest's electrical bioimpedance (EBI). This increase in EBI can be measured non-invasively using impedance pneumography (IP) and correlates strongly (r>0.9) with changes in lung volume (TV). Further, IMU-based acceleration sensing provides a complementary measurement of external chest wall movement during respiration. IMU sensors can track lateral expansion, which correlates strongly (r>0.9) with changes in TV. Additionally, surface electromyography (sEMG) (as used in devices and systems as provided herein) enables non-invasive measurement of EAdi, which is typically obtained invasively through esophageal recording at the diaphragmatic level.

In alternative embodiments, devices and systems and methods as provided herein can monitor diaphragmatic efficiency in individuals with Cervical Spinal Cord Injury (CSCI), which can prevent respiratory complications. Respiratory complications related to CSCI are a leading cause of death due to the innervation of the diaphragm by the phrenic nerve originating from the third to fifth cervical roots.

In alternative embodiments, devices and systems and methods as provided herein monitor diaphragmatic efficiency in individuals in need thereof and can add another dimension of respiration health information to existing metrics, allowing a better titration of treatment (e.g., amount of ventilation at home).

In alternative embodiments, provided are wearable diaphragmatic efficiency monitoring methods, systems, and devices that may be used to measure and monitor diaphragmatic efficiency, and in alternative embodiments, allow local (e.g., mobile or in-home) respiratory monitoring of people with respiratory diseases or conditions including, for example, cervical spinal cord injury (CSCI), asthma, chronic obstructive pulmonary disease (COPD), sleep apnea, patients undergoing positive airway pressure (PAP) testing, or PAP-NAP studies. In alternative embodiments, provided are products of manufacture and kits, comprising portable, wearable diaphragmatic efficiency monitoring devices, and methods for using them.

In alternative embodiments, provided are wearable diaphragmatic efficiency monitoring technologies and devices that can significantly improve early detection rates of respiratory dysfunction; where the wearable diaphragmatic efficiency monitoring technologies and devices as provided herein can provide a precise and cost-effective wearable sensor system that can objectively assess diaphragmatic efficiency. In alternative embodiments, this may be achieved through the monitoring of the amount of air exchanged in the individuals in need thereof during each breath and monitoring of the electrical activity of their diaphragm.

In alternative embodiments, provided are unobtrusive sensor systems that can reliably monitor diaphragmatic efficiency locally to where an individual in need may be present, e.g., in the home, hospital, hospice, or anywhere in the community. In alternative embodiments devices and systems as provided herein allow continual and accurate monitoring of diaphragmatic health and significantly increase early detection rates of serious respiratory complications in cervical spinal cord injury (CSCI) patients. In alternative embodiments, devices and systems as provided herein can also be used in other patient populations with chronic respiratory issues to titrate their treatment.

To better measure NVE locally (as opposed to at, say, a clinic), e.g., at home, devices and systems and methods as provided herein comprise a portable, e.g., wearable, hardware capable of capturing multiple noninvasive sensing modalities or devices, including impedance pneumography (IP), inertial measurement unit (IMU), and surface electromyography (sEMG). Benefits of these sensing modalities and combinations include:
IP: As air enters the lungs, tissue conductivity decreases, leading to an increase in chest's EBI. This increase in EBI can be measured noninvasively using IP and correlates strongly (r>0.9) with changes in lung volume (TV), as discussed above.
IMU: IMU-based acceleration sensing provides a complementary measurement of external chest wall movement during respiration. IMU sensors can track lateral expansion, postural shifts, and motion artifacts, offering critical context when interpreting IP-derived signals. Because IP is sensitive to electrode position and posture, and IMU may overestimate or underestimate movement during paradoxical or shallow breathing, the combination enables cross-validation of TV estimates. This synergistic approach improves detection of inefficient or compensatory breathing patterns that would not be evident from either signal alone.
sEMG: Surface electromyography (sEMG) enables non-invasive measurement of EAdi, which is typically acquired invasively through esophageal recording. sEMG can quantify respiratory muscle recruitment, including diaphragm and accessory muscle activity, enabling estimation of neural respiratory drive.

In certain embodiments, combining IP and IMU signals allows for correction of postural or motion-induced distortion in TV estimation and improves system wearability. For example, integrating IP and IMU sensors into a modular, partial-coverage wearable (as nonlimiting examples, an abdominal binder or flexible chest strap) can reduce the need for full circumferential sensor placement, enhancing ease of use and user compliance.

The captured IP, IMU, and sEMG signals may be processed by a processor to extract TV and EAdi, from which neuro-ventilatory efficiency (NVE) may be calculated, e.g., as TV/EAdi.

In alternative embodiments, the system further extracts one or more derived metrics such as but not limited to neck-to-diaphragm sEMG ratio, which reflects compensatory use of accessory muscles, with elevated values indicating diaphragm fatigue or dysfunction.

Provided are products of manufacture (for example, the wearable diaphragmatic efficiency monitoring technologies and devices as provided herein) and kits for practicing methods as provided herein; and optionally, products of manufacture and kits can further comprise instructions for practicing methods as provided herein.

Example devices and systems can provide a precise and cost-effective wearable sensor system that can objectively and unobtrusively assess diaphragmatic efficiency. This may be achieved through the monitoring of tidal volume (TV) using impedance pneumography (IP) and IMU signals, and the monitoring of the electrical activity of the diaphragm (EAdi) using surface electrical myography (sEMG). In alternative embodiments, devices and systems and methods as provided herein are capable of simultaneously capturing IP, IMU, and sEMG signals, and also may incorporate one or more algorithms that can accurately predict neuro-ventilatory efficiency (NVE) using TV obtained from IP and IMU, and electrical activity of diaphragm (EAdi) from sEMG.

In alternative embodiments, devices, systems, and/or methods as provided herein can provide continual and accurate monitoring of diaphragmatic health and significantly increase early detection rates of serious respiratory complications. In alternative embodiments, devices, systems, and/or methods as provided herein can provide an accurate and cost-effective wearable sensor system that objectively and unobtrusively assesses diaphragmatic efficiency of CSCI patients living in a community.

In alternative embodiments, devices and systems and methods as provided herein can provide high signal-to-noise-ratio (SNR) IP and sEMG recordings for accurate diaphragmatic efficiency, and additionally or alternatively may provide an unobtrusive and comfortable system that allows for long-term operation without frequent charging.

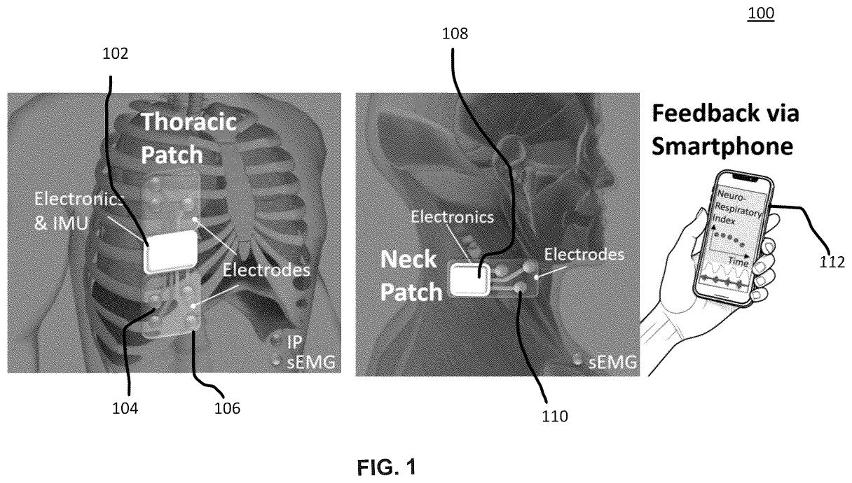

Referring now to the drawings, FIG. 1 illustrates an example noninvasive wearable system 100 including two example application-specific modules, embodied in a flexible thoracic patch 102, with one or a plurality of electrodes fabricated for attachment to the thorax (chest) for each of IP detection (electrodes 104) and sEMG detection (electrodes 106), and in a neck patch 108 with one or a plurality of electrodes 110 fabricated for attachment to the neck for sEMG detection.

In the example noninvasive wearable system 100 the thoracic patch 102 monitors diaphragm activity, tidal volume, and chest movement, and the neck patch 108 monitors neck muscle activity. Signals generated using the patches 102, 108, e.g., directly, after suitable processing and/or amplification, etc., may be transmitted, e.g., wirelessly, to a processor-based device such as but not limited to a smartphone 112, where they are processed to extract neuro-respiratory indices.

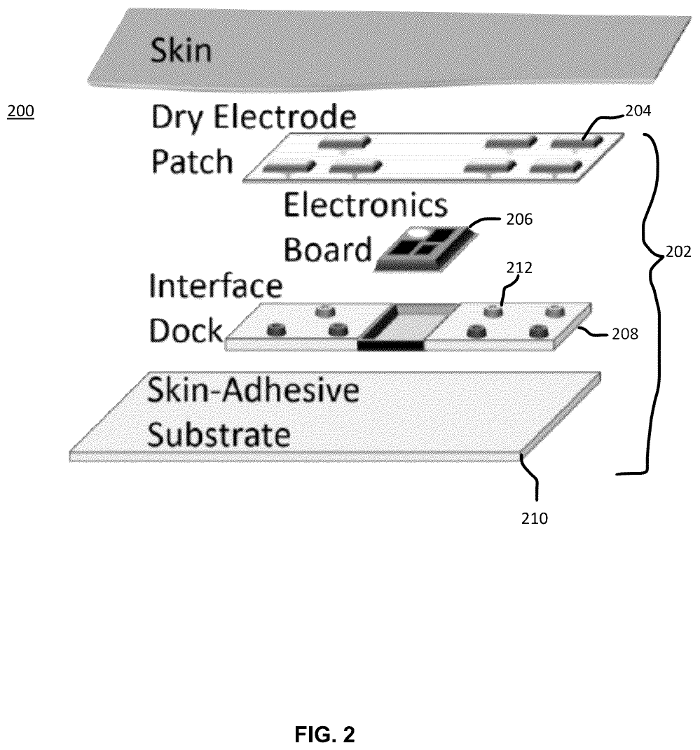

FIG. 2 shows an example patch 200, which may correspond to thoracic patch 102 or neck patch 108. The patch 200 may include a multi-layer (e.g., four-layer) stack 202 that can separate reusable and disposable components while enabling relatively secure and effortless reassembly, e.g., using magnetic docking.

As shown in FIG. 2, example hardware modules such as patch 200 can be organized into multiple layers including a dry electrode patch 204, a (e.g., reusable) electronics board 206, a central interface dock 208, and a skin-adhesive substrate 210. Dry electrodes for IP and sEMG may be embedded into the flexible dry electrode patch 204 and their signals routed to exposed contact pads. The dry electrode patch 204 may be magnetically docked onto the electrode interface mount 206, e.g., using low-profile neodymium magnets, to ensure precise mechanical alignment and maintain consistent electrical contact with female snaps 212 located on the mount. The example patch 200 may be configured to be pre-assembled, pre-routed, and replaced regularly (e.g., daily), with no lead manipulation required by the user.

An example reusable Electronics Board 206 includes a microcontroller, an analog front-end, inertial measurement unit (IMU), Bluetooth Low Energy (BLE) module, and a power source, which may be embodied in a battery such as a coin cell battery (e.g., CR2032) supporting a substantial duration, e.g., >72 hours, of continuous use. The example board 206 may be housed in a compact, low-profile enclosure that magnetically aligns with the electrode interface, e.g., using pogo pin contacts or other suitable contacts. This can provide easy docking without fine motor control, thereby further supporting independent use. IMU-based motion detection can be incorporated, for instance, to pause data collection during high-motion periods and resume during rest. Wear-state detection and real-time signal quality checks can be provided to confirm electrode contact and flag dropouts. Data may stream via wireless transmission methods such as but not limited to Bluetooth low energy (BLE) to a processor-based device such as but not limited to paired smartphone, e.g., smartphone 112 for logging, visualization, background uploads, etc. An application, e.g., a mobile app, can be provided on the smartphone 112 or other device for, e.g., installed in or accessed by, the processor-based device to manage pairing, manage session control, and/or for long-term storage.

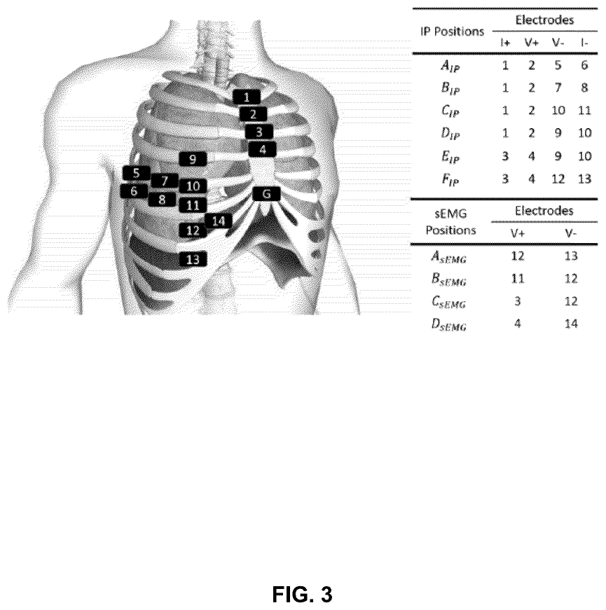

FIG. 3 illustrates exemplary positions of electrodes on the thorax of an individual, where the electrodes are used in devices and systems disclosed herein.

In example embodiments, a smartphone app can be configured to be user-friendly and provided as a primary interface between the wearable hardware (e.g., patches 102, 108), individuals in need (e.g., patients), and clinicians. A nonlimiting example application may be built on the React Native platform [32], support mobile operating systems such as iOS and Android, and be configured to, among other features, integrate seamlessly to receive IP, sEMG, and IMU data in real time. Optionally, to provide greater accessibility for users with limited finger control or cognitive energy, the example app can include voice control or similarly beneficial interface for key functions such as session start, symptom logging, and/or navigation. An example app may alternatively or additionally feature automated session initiation, wear-time logging, and/or real-time alerts for signal loss or sensor detachment. An (e.g., minimal-touch) interface can be configured to display one or more of session status, signal quality, and posture classification to support engagement and self-monitoring. A history module may be provided to allow users to track trends in estimated TV, muscle activity ratios, and duty cycle, promoting awareness and adherence while providing clinicians with objective remote data. To support symptom tracking, a user interface such as but not limited to a voice-enabled daily questionnaire can be provided to allow hands-free logging of breathing symptoms, fatigue, and health events.

The invention will be further described with reference to the examples described herein; however, it is to be understood that the invention is not limited to such examples.

### EXAMPLES

### Exemplary Devices

In alternative embodiments, an electronics board 206 for IP detection systems (IP systems) used in devices and systems as provided herein can include a front-end chip such as an AD5940™ (Analog Devices, Wilmington, MA) impedance front-end [35], which captures the resistive component of the electrical bioimpedance (EBI), strongly correlated to lung volume [36], [37], across frequencies (e.g., up to 200 kHz) within the impedance pneumography frequency range [38]. The example chip can allow control of injected current signal amplitude to meet safety standards (IEC 60601-1) and optimize SNR within the dynamic range.

In alternative embodiments, an electronics board 206 for an exemplary sEMG system may include an ADS1198™ (Texas Instruments, Dallas, TX) analog front-end [39]. This example analog front-end can employ active grounding, high common-mode rejection ratio instrumentation amplifier, and gain control to optimize sEMG signal amplification without saturation.

As an example of IMU selection, miniaturized MEMS units may be selected. To improve robustness, an IMU combining accelerometer and gyroscope may be provided. While the accelerometer may be the primary sensor used to capture linear thoracic movement due to breathing, the gyroscope can provide angular velocity data that can distinguish true respiration-induced motion from postural shifts. Based on literature [33], [34], respiratory-induced motion typically produces accelerations less than a few hundreds of milli-g range. To achieve sufficient resolution (e.g., >10 bits), selected IMUs can be configured, as a nonlimiting example, to have a noise floor below 100 μg/√Hz and full-scale ranges at a maximum of ±2 g. An example IMU may be ultra-low power, achieving those specs within the respiratory bandwidth of <3 Hz (e.g., ADXL326, BMI160).

In alternative embodiments, analog front-end integrated electronics, which may be custom-designed or otherwise configured, can be used for example electronics boards 206 to capture sEMG and IP. These designs can include off-the-shelf instrumentation amplifiers, operational amplifiers, passive circuit elements, and peripheral electronics. For instance, in alternative embodiments a minimum detectable signal of 55 μΩrms may be achieved ([40], [41]). Example designs may also include compact transistor-level integrated circuits developed using state-of-the-art technology nodes, a nonlimiting example being 65 nm CMOS or 22 nm CMOS.

In alternative embodiments, wet-gel electrodes and dry electrodes, e.g., fabric electrodes, metallic electrodes, foam-based electrodes, polymer-based electrodes, etc. can be used to interface the electronics with the skin (e.g., for electrodes 104, 106, 110, 204).

In alternative embodiments, the captured sEMG and IP signals can be digitized, e.g., on the electronics board 206, by an analog-to-digital converter (ADC) and logged via off-the-shelf data logging systems onto an onboard storage medium, such as a micro-SD card.

In alternative embodiments, the analog or digitized sEMG and IP signals can be transferred, e.g., from the electronics board 206, to a remote processor via Bluetooth or similar wireless data transfer technology.

In other embodiments, the captured sEMG and IP signals can be processed either onboard or remotely by a commercial or custom-designed microprocessor, e.g., on the electronics board 206, to extract important features for monitoring tidal volume (TV) and electrical activity of the diaphragm (EAdi).

In alternative embodiments, the integration of impedance pneumography (IP) and IMU signals for TV estimation may follow any of various processing strategies. In some cases, IP and IMU signals may first be independently cleaned and then merged through sensor fusion methods such as Kalman filtering, complementary filtering, or weighted averaging, while in other cases fusion may be performed at the feature level by combining extracted amplitudes, durations, and rates prior to modeling. After fusion, outlier detection and removal may be performed using statistical thresholds, median absolute deviation, or dynamic modeling to improve robustness. The resulting waveform features may then be mapped to TV using regression models, which may include linear regression, polynomial regression, or machine learning approaches such as neural networks or ensemble models. Calibration may be performed on a subject-specific basis, or alternatively a generalized calibration model may be adapted to new subjects using transfer learning or adaptive updating of regression coefficients.

In alternative embodiments, the processed TV and EAdi can be used to calculate the neuro-ventilatory efficiency (NVE), e.g., as TV/EAdi. The calculated NVE can then be stored and monitored longitudinally. Any significant changes in the data can trigger alerts for the user or authorized individuals if a critical threshold is reached.

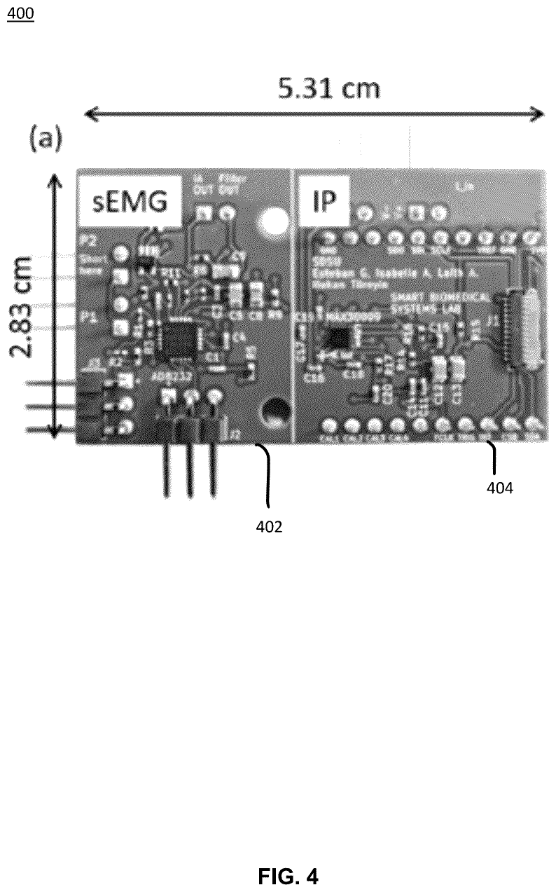

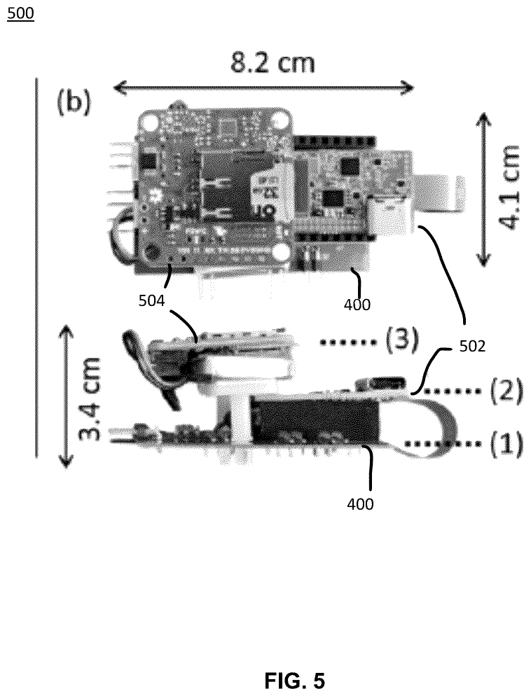

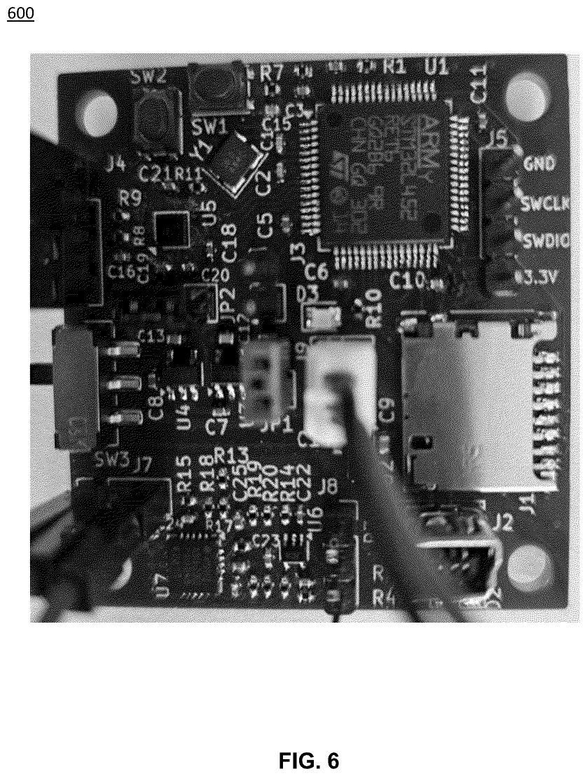

FIG. 4 shows an example sensor board 400 integrating IP 402 and sEMG 404 sensor electronics on the (here, single) compact board. It will be appreciated that the scale is provided merely to illustrate example dimensions for the sensor board 400, and other sizes can be provided. FIG. 5 shows an example complete stack 500 including the sensor board 400 in FIG. 4 (1), stacked with the example IP-MCU board (2) 502, and a data logger (3) 504 embodied in OpenLog Artemis data logger (Sparkfun, Boulder, CO), which samples at 1.9 kHz and includes a world-time clock for synchronization with IP data. The example board 400 is powered by 1.8V from the IP-MCU for IP blocks and 3.3V from the OpenLog Artemis for sEMG blocks. FIG. 6 shows another example layout for a circuit board 600 illustrating integrated IP, sEMG, data acquisition, and power management electronics.

### Experiments

In experiments using example sensor hardware, IP and sEMG data were collected during shallow and deep breathing at different MIP levels. Measurements were taken from a convenience sample of eight healthy young male adults (age: 23±1.9 years, height: 177.8±8.5 cm, weight: 76.7±10.7 kg) with no known respiratory or cardiovascular issues, while seated comfortably in an upright position. Two data collection sessions were conducted to identify optimal electrode positions for measuring TV using IP and EAdi using sEMG. Based on previous work, e.g., [42]-[45], six IP and four sEMG positions were selected, as summarized in FIG. 3, where G is the sEMG ground electrode.

In a first session, IP electrode positions were evaluated for TV and RR accuracy using a Biopac TSD117B flowmeter with nostrils clipped. Subjects completed 60 s baseline, followed by 120 s shallow and deep breathing trials with 30 s rests. A second session assessed sEMG electrode positions by correlating sEMG amplitude with breathing effort. MIP was measured via maximal inhalation against a blocked airway using a K5 IMT device (POWERBreathe, UK). Subjects performed 30 s breathing exercises at 10-60% MIP in 10% increments, with 30 s rest periods. All measurements were repeated for each electrode position.

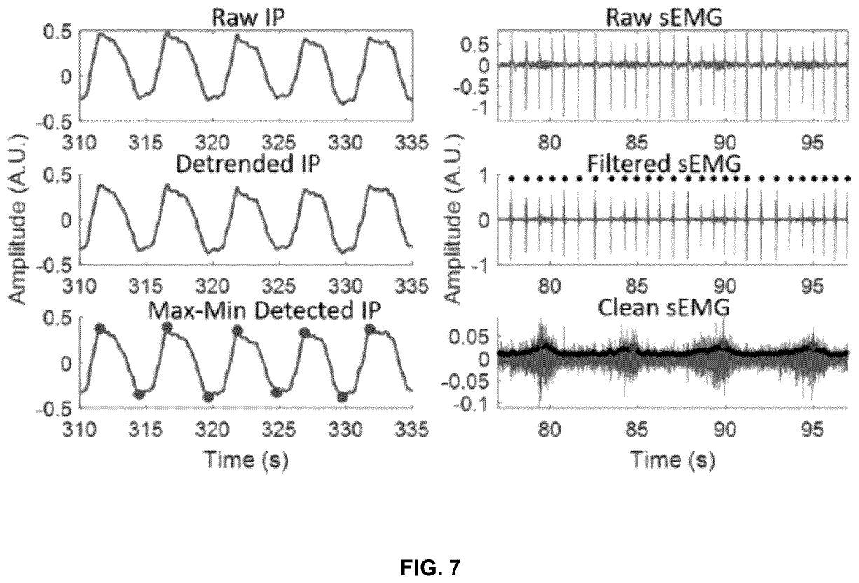

Signal processing and analysis were conducted in MATLAB (Math Works, Natick, MA). FIG. 7 shows example waveforms from the signal processing chain, where IP waveforms (FIG. 7, left) were detrended using a second-order polynomial, and peak-to-peak amplitudes (IPₚₚ) were calculated. Instantaneous respiration rate (RRₘₑₐₛ) (FIG. 7, right) was determined from time differences between successive peaks. The IP panels show raw, detrended, and peak-detected signals. sEMG panels show raw, filtered, and SVD-cleaned signals with RMS (black) and peaks (red).

True tidal volume (TVₜᵣᵤₑ) was calculated by integrating airflow data from the flowmeter, converting airflow to volume. Step sizes in the resulting volume waveform represented TVₜᵣᵤₑ for each breath. Time differences between peaks of the true air volume waveform were used to calculate the true instantaneous respiration rate (RRₜᵣᵤₑ).

The sEMG waveforms were processed using a fourth-order Butterworth band-pass filter (10-400 Hz) to remove baseline wander and high-frequency noise, followed by a second-order notch filter (57-63 Hz) to eliminate power line interference. Significant electrocardiogram (ECG) artifacts were removed using singular value decomposition (SVD) (E. Peri et al., “Singular value decomposition for removal of cardiac interference from trunk electromyogram,” Sensors, vol. 21, no. 2, p. 573, January 2021). ECG peaks were first detected using the Pan-Tompkins algorithm (Complete Pan Tompkins Implementation ECG QRS Detector, Accessed: Dec. 28, 2024, https://www.mathworks.com/matlabcentral/fileexchange/45840-complete-pan-tompkinsimplementation-ecg-qrs-detector), and the sEMG signal was segmented around each QRS complex (0.3 s before the QRS wave, with a window length equal to the median QRS interval). A matrix X was constructed with rows representing QRS segments, and SVD was applied to obtain singular vectors (SVs). The ECG component was reconstructed using inverse SVD on n SVs (where n, ranging from 8 to 20, was automatically selected to maximize the SNR of the cleaned sEMG). The reconstructed ECG was subtracted from the filtered sEMG to yield the cleaned sEMG. Finally, the root-mean-square peak amplitudes of the cleaned sEMG (sEMG_{RMS,p}) were calculated.

For IP positions, TV and RR estimation accuracies were assessed using linear regression between IPₚₚ and TVₜᵣᵤₑ to compute TV_{pred}. R², mean absolute percentage error (MAPE), and root-mean-square error (RMSE) between TVₜᵣᵤₑ and TV_{pred} across positions were compared using one-way ANOVA. For RR estimation, RMSE, MAPE, and mean absolute error (MAE) were calculated and analyzed similarly. For sEMG, the correlation between sEMG_{RMS,p} and MIP percentages was assessed using R² from linear regression, followed by one-way ANOVA comparison.

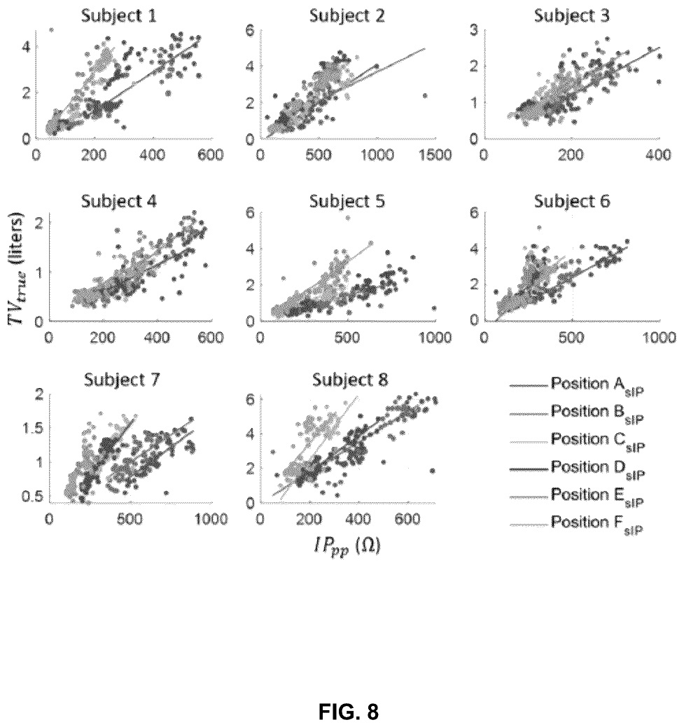

Analysis for experimental IP measurements is as follows: Scatter plots of IPₚₚ vs. TVₜᵣᵤₑ with linear regression curves for all subjects and positions are shown in FIG. 8. Table 1, below, summarizes R², MAPE, and RMSE for TV estimation across positions. All positions show a positive correlation between TVₜᵣᵤₑ and IPₚₚ, with R² values ranging from 0.45 (S8, Position FIP) to 0.97 (S1, Position F_{IP}). Average R² values for all positions exceeded 0.69. Position C_{IP}, between the upper sternum and the midclavicular line (MCL) at the 4th intercostal (IC) space, provided the highest correlation with true tidal volume, with the highest average R² (0.79±0.13), the lowest RMSE_{TV} (0.33±0.15 L), and a low MAPE_{TV} (17.89±4.91%). Position C_{IP}, located between the upper sternum and the MCL at the 5th IC space, followed closely, with R²=0.77±0.08, MAPE_{TV}=17.59±5.30%, and consistent results. Position A_{IP}, located between the upper sternum and the anterior axillary line (AAL) at the 5th IC space, performed the worst, with the lowest R² (0.69±0.14), the highest MAPE_{TV} (21.74±6.91%), and a relatively high RMSE_{TV} (0.42±0.20 L). Subject-specific variations were noted, with some (e.g., S1 and S6) achieving consistently high R² values, while others (e.g., S7) showed lower values.

TABLE 1

| | | RMSE_{TV} | MAPE_{TV} |
|---|---|---|---|
| Position | R² | (L) | (%) |
| A_{IP} | 0.69 ± 0.14 | 0.42 ± 0.20 | 21.74 ± 6.91 |
| B_{IP} | 0.70 ± 0.17 | 0.41 ± 0.24 | 22.82 ± 13.12 |
| C_{IP} | 0.77 ± 0.08 | 0.34 ± 0.18 | 17.59 ± 5.30 |
| D_{IP} | 0.79 ± 0.13 | 0.33 ± 0.15 | 17.89 ± 4.91 |
| E_{IP} | 0.76 ± 0.15 | 0.34 ± 0.20 | 17.95 ± 7.32 |
| F_{IP} | 0.76 ± 0.18 | 0.35 ± 0.25 | 17.67 ± 3.92 |

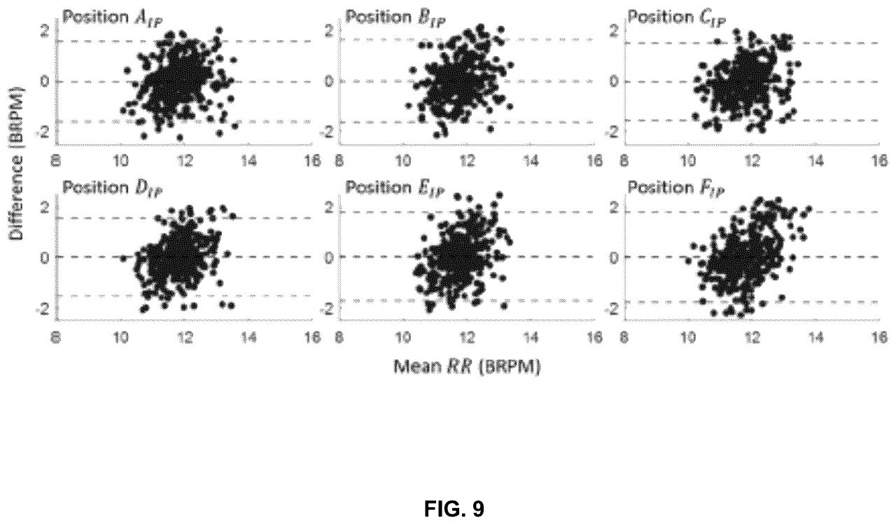

For RRₘₑₐₛ (Table 2, shown below), Position C_{IP} was most accurate and consistent, with the lowest RMSE_{RR} (0.76±0.25 BRPM), MAPE_{RR} (5.16±1.75%), and MAE_{RR} (0.61±0.20 BRPM). Position A_{IP} also performed well, while Positions E_{IP} and F_{IP}, the only two positions with upper electrodes placed on the mid-sternum instead of the upper sternum, exhibited greater errors and variability. Bland-Altman plots (FIG. 9) show mean errors near zero and 95% confidence intervals of approximately ±2 BRPM, and thus indicating IP is reliable for RR measurement across positions. In FIG. 9 shows differences between RRₘₑₐₛ and RRₜᵣᵤₑ across different positions, where black dashed line indicates mean difference, and red dashed lines indicate 95% confidence intervals.

TABLE 2

| | RMSE_{RR} | MAPE_{RR} | MAE_{RR} |
|---|---|---|---|
| Position | (BRPM) | (%) | (BRPM) |
| A_{IP} | 0.79 ± 0.12 | 5.34 ± 0.83 | 0.63 ± 0.10 |
| B_{IP} | 0.88 ± 0.42 | 6.09 ± 3.09 | 0.71 ± 0.35 |
| C_{IP} | 0.76 ± 0.25 | 5.16 ± 1.75 | 0.61 ± 0.20 |
| D_{IP} | 0.84 ± 0.14 | 5.52 ± 0.80 | 0.65 ± 0.10 |
| E_{IP} | 0.89 ± 0.31 | 6.20 ± 2.47 | 0.71 ± 0.24 |
| F_{IP} | 0.90 ± 0.23 | 5.89 ± 1.50 | 0.70 ± 0.18 |

Correlations between sEMG_{RMS,p} and M_{IP} across all electrode positions and subjects showed wide variability (R²=0.015 to 0.944). Position C_{sEMG}, located between the mid-sternum and the MCL at the 6th IC, demonstrated the best performance and exhibited the highest average R² (0.69±0.28) with the lowest variability, while Position A_{sEMG}, located between the MCL at the 6th and 7th IC spaces, had the lowest average R² (0.54±0.32) and the highest variability.

One-way ANOVA (80% confidence) showed no significant differences in R², RMSE_{TV}, or MAPE_{TV} across IP electrode positions (A-F) and no significant differences in R² across sEMG electrode positions (A-D).

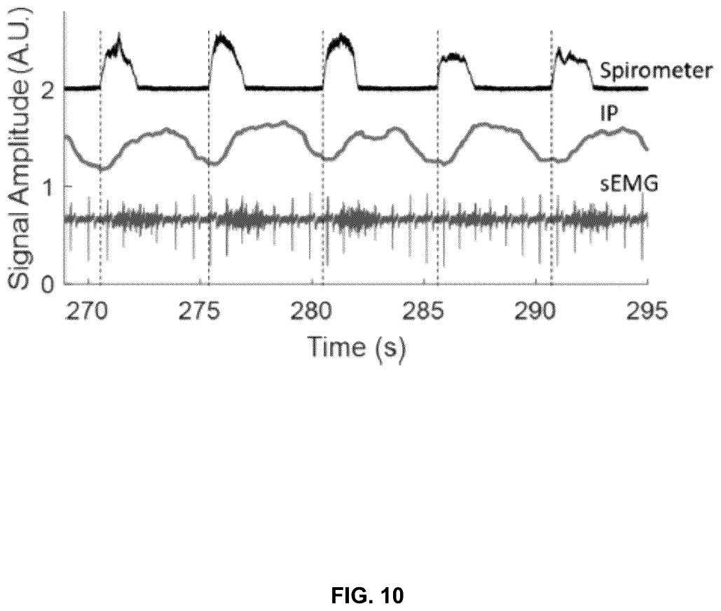

The example hardware was evaluated for power consumption and data collection. The integrated IP and sEMG sensor board consumed a total of 2.17 mW, with the IP sensor using 0.25 mA at 1.8 V (0.45 mW) and the sEMG sensor using 0.51 mA at 3.3 V (1.72 mW). This power efficiency allows the example sensor board (excluding data logger boards) to operate for over 130 hours with a 105 mAh LiPo battery. Simultaneous data collection from the IP and sEMG sensors, along with airflow data from a flowmeter, was performed on one participant and is shown in FIG. 10, uncorrected for ECG. Measurements were taken at the positions with the highest average R² from earlier hardware analysis (D_{IP} and C_{sEMG}). These results demonstrate an example system's capability for simultaneous signal acquisition.

The analysis shows strong correlations between TVₜᵣᵤₑ and TV_{pred}, with some variability across IP positions, and consistent accuracy in IP-based RR measurements across positions. However, correlations between sEMG and MIP exhibit significant variability. No electrode position for IP or sEMG proved statistically superior, suggesting precise placement may not be critical, which can simplify integration into example settings.

### Example Benefits and Applications

Example devices, systems and methods provide a novel noninvasive and wearable respiratory monitoring device. Example IP and sEMG sensors can be configured, e.g., using selected and configured analog front-ends, to meet available safety standards. In some example operations, measurement of TV with pneumotachography and EAdi with esophageal balloon catheterization may be used to establish optimal electrode placement and develop one-time, subject-specific calibration algorithms. Testing in individuals with CSCI may be performed to assess dry-electrode designs and fine-tuning electrode placement.

Extended home monitoring may be provided, e.g., via smartphone or other processor-based device integration, with algorithms configured to minimize long-term monitoring-related artifacts such as motion-artifact removal and real-time contact monitoring with pre-/post-monitoring NVE assessments performed, e.g., in a clinic.

In general, example hardware and methodologies provided herein can enable more frequent outpatient monitoring and early intervention for respiratory function, enhancing patient care. Results demonstrate usefulness for people with CSCI, who initially have respiratory failure requiring mechanical ventilation; once weaned from the ventilator, their pulmonary function is rarely monitored until they gradually develop an issue. The development of a portable system enabling concurrent IP and diaphragm sEMG measurements offers promise for more frequent, effective respiratory monitoring that can facilitate better management of respiratory conditions, enhancing patient care and quality of life.

### General

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

A number of embodiments of the invention have been described. Nevertheless, it can be understood that various modifications may be made without departing from the spirit and scope of the invention. Accordingly, other embodiments are within the scope of the following claims.

What is claimed is:

1. A product of manufacture for measuring and monitoring diaphragmatic efficiency in an individual in need thereof, comprising a wearable hardware comprising noninvasive sensing devices, components, or modalities, the product of manufacture comprising:
(a) a hardware impedance pneumography (IP) device comprising one or a plurality of electrodes fabricated for attachment to the thorax (chest) of the individual in need thereof;
(b) an inertial measurement unit (IMU) device for attachment to the thorax (chest) of the individual in need thereof; and
(c) a surface electromyography (sEMG) device comprising one or a plurality of electrodes fabricated for attachment to the thorax (chest) or the neck of the individual in need thereof,
wherein the IP, IMU, and sEMG devices are operably connected to:
(i) a power source and power management electronics, and
(ii) a data transmittal device for transmitting IP, IMU, and sEMG device signals or diaphragmatic measurement data produced from such signals to a remote processor-based device.

2. The product of manufacture of claim 1, wherein all or separately each of the product of manufacture components are part of, or adhered to, a skin wearable patch.

3. The product of manufacture of claim 1, wherein IP, IMU, and sEMG signals are linear filtered to suppress noise sources, wherein optionally the noise sources comprise high-frequency noise, baseline wander and/or electrocardiogram interference.

4. The product of manufacture of claim 1, wherein the IP device further comprises a processor configured for phase-sensitive detection for enhanced signal-to-noise-ratio (SNR) by amplifying the narrow bandwidth and suppressing out-of-band noise.

5. The product of manufacture of claim 4, wherein the processor is further configured for controlling injected current signal amplitude to meet one or more safety standards and optimize SNR within a dynamic range.

6. The product of manufacture of claim 1, wherein the sEMG device further comprises a high common-mode rejection ratio instrumentation amplifier and gain controller to optimize sEMG signal amplification without saturation.

7. The product of manufacture of claim 1,
wherein optionally the remote device comprises a remote computer, cloud storage, tablet or cell phone; and
wherein the data transmittal device transmits the IP, IMU, and sEMG device signals or diaphragmatic measurement data produced from such signals wirelessly.

8. The product of manufacture of claim 1, wherein the IP device is further configured to detect acceleration for use in interpreting IP-derived signals.

9. The product of manufacture of claim 1, wherein the IP, IMU, and sEMG devices each comprise a hardware stack including an electronics board, a dry electrode patch, and a skin-adhesive substrate.

10. The product of manufacture of claim 9, wherein the hardware stack further includes an interface dock for interfacing with the electronics board and the dry electrode patch.

11. A method for detecting and measuring and monitoring diaphragmatic efficiency in an individual in thereof comprising:
(a) providing a product of manufacture of claim 1, and attaching electrodes to the individual in need thereof; and
(b) using the IP, IMU, and sEMG devices taking or reading diaphragmatic measurement data from the individual in need thereof, and
(c) transmitting the diaphragmatic measurement data to the remote device, wherein optionally the remote device comprises a remote computer, cloud storage, tablet or cell phone.

12. The method of claim 11, wherein the individual in thereof has a respiratory disease or condition, or the individual in thereof is a patient undergoing positive airway pressure (PAP) testing, or PAP-NAP studies.

13. The method of claim 12, wherein the respiratory disease or condition comprises: a cervical spinal cord injury (CSCI), asthma, chronic obstructive pulmonary disease (COPD), and/or sleep apnea.

14. The method of claim 11, further comprising:
use of a Savitzky-Golay filter to clean the IP signal and generate a cleaned IP signal to mitigate anticipated cardiogenic oscillation interferences.

15. The method of claim 14, further comprising analyzing the cleaned IP signal to determine respiration phases, durations, respiration rates, and/or peak-to-peak amplitude changes.

16. The method of claim 11, wherein the sEMG device further comprises a high common-mode rejection ratio instrumentation amplifier and gain controller to optimize sEMG signal amplification without saturation.

17. The method of claim 11, further comprising calculating diaphragmatic efficiency using the diaphragmatic measurement data.

18. Use of a product of manufacture of claim 1 for measuring and monitoring diaphragmatic efficiency in an individual in need thereof, wherein optionally the individual in thereof has a respiratory disease or condition, or the individual in thereof is a patient undergoing positive airway pressure (PAP) testing, or PAP-NAP studies, and optionally the respiratory disease or condition comprises: a cervical spinal cord injury (CSCI), chronic obstructive pulmonary disease (COPD), asthma and/or sleep apnea.

19. A product of manufacture of claim 1 for use in measuring and monitoring diaphragmatic efficiency in an individual in need thereof, wherein optionally the individual in thereof has a respiratory disease or condition, or the individual in thereof is a patient undergoing positive airway pressure (PAP) testing, or PAP-NAP studies, and optionally the respiratory disease or condition comprises: a cervical spinal cord injury (CSCI), chronic obstructive pulmonary disease (COPD), asthma and/or sleep apnea.

20. A kit comprising a product of manufacture of claim 1.
